(19) United States
(12) Patent Application Publication
(10) Pub. No.: US 2010/0174279 A1
(43) Pub. Date: Jul. 8, 2010

(54) RADIOFREQUENCY THERMAL BALLOON CATHETER SYSTEM

(76) Inventor: Shutaro SATAKE, Kamakura-shi (JP)

Correspondence Address: BIRCH STEWART KOLASCH & BIRCH, PO BOX 747, FALLS CHURCH, VA, 22040-0747, US

(21) Appl. No.: 12/491,902

(22) Filed: Jun. 25, 2009

(30) Foreign Application Priority Data
Jan. 4, 2009 (JP) 2009-088890

(51) Int. Cl. A61B 18/08

(52) U.S. Cl. 606/28

(57) ABSTRACT

A radiofrequency thermal balloon catheter system provided with a vibration generator that enables the arbitrary setting of the amplitude of a vibrational wave, and is less damageable, and capable of generating powerful vibrational waves. An external vibration generator is equipped with an injector and a piston driven by a crank coupled to a rotating disk to reciprocate. Therefore, the external vibration generator can arbitrarily set the amplitude of the vibrational wave by reciprocating an injector inner tube by virtue of the piston to adjust the amplitude and cycle of the vibrational wave depending on a bore diameter of the injector and the rotating speed of a rotating disk. Hence, there can be provided the radiofrequency thermal balloon catheter system which is equipped with the vibration generator that is capable of exercising the above performance, less damageable and can generate the powerful vibrational waves.

### BACKGROUND OF THE INVENTION

1. Field of the invention

The present invention relates to a radiofrequency thermal balloon catheter system, particularly to a radiofrequency thermal balloon catheter system used to treat cardiovascular diseases.

2. Description of related Art

In a radiofrequency thermal balloon catheter system, in order to agitate a solution inside a balloon to keep a temperature of the solution uniform, a vibration generator for applying vibrational waves to an inside of the balloon has been used. As the conventional vibration generator like this, a vibration generator remodeled from a roller pump has been known as disclosed in, e.g., Japanese Unexamined Patent Application Publication No. 2005-185661. The vibration generator disclosed therein utilizes elasticity of a coupling tube. Accordingly, a filling solution is driven out by compressing the coupling tube with a roller, while the filling solution is drawn in by ceasing the compression to restore the tube by its own elasticity. Repeating these alternate operations enables the vibrational waves to be sent into the balloon.

This structure, however, had disadvantages that an amplitude of the vibrational wave was not arbitrarily settable and as being subjected to a hard rubbing action by the roller, the coupling tube was easily damaged. Further, the structure had a defect that due to utilizing the elasticity of the coupling tube for drawing in the filling solution, powerful vibrational waves could not be generated.

### SUMMARY OF THE INVENTION

With the view of the problems described above, it is, therefore, an object of the present invention to provide a radiofrequency thermal balloon catheter system equipped with a vibration generator which enables amplitudes of vibrational waves to be arbitrarily set and further is not easily damaged and can generate powerful vibrational waves.

A first aspect of the present invention is a radiofrequency thermal balloon catheter system comprising: a catheter shaft comprising an outer tube and an inner tube which are slidable to each other; a balloon provided between a distal end of said outer tube and a vicinity of a distal end of said inner tube; an electrode for delivery of radiofrequency current provided in a central portion of said balloon, an external radiofrequency generator which supplies radiofrequency energy to said electrode for delivery of radiofrequency current; a temperature sensor provided inside said balloon; an external thermometer which detects temperature of said temperature sensor; a solution transport path formed between said outer tube and said inner tube, in communication with an inside of said balloon; an external vibration generator which applies vibrational waves to the inside of said balloon through said solution transport path; and a guide wire which guides said balloon to a target site, wherein said external vibration generator includes an injector comprising an injector outer tube fixed inside a cylinder and an injector inner tube inserted into said injector outer tube, and a piston which is slidably provided inside said cylinder and is driven by a crank coupled to a rotating disk to reciprocate. In said external vibration generator, said injector inner tube is fixed to said piston and therefore a forward motion of said piston allows said injector inner tube to be pushed to thereby drive out a filling solution in said injector into said solution transport path, causing said balloon to dilate, while a backward motion of said piston allows said injector inner tube to be pulled to thereby draw a filling solution in said solution transport path into said injector, causing said balloon to contract. Besides, the amplitude and cycle of said vibrational wave are arbitrarily adjustable depending on a bore diameter of said injector and a rotating speed of said rotating disk.

A second aspect of the present invention is a radiofrequency thermal balloon catheter system comprising: a catheter shaft comprising an outer tube and an inner tube which are slidable to each other; a balloon provided between a distal end of said outer tube and a vicinity of a distal end of said inner tube; an electrode for delivery of radiofrequency current provided in a central portion of said balloon; an external radiofrequency generator which supplies radiofrequency energy to said electrode for delivery of radiofrequency current; a temperature sensor provided inside said balloon; an external thermometer which detects temperature of said temperature sensor; a solution transport path which is formed between said outer tube and said inner tube, in communication with an inside of said balloon; an external vibration generator which applies vibrational waves to the inside of said balloon through said solution transport path; and a guide wire which guides said balloon to a target site, wherein said external vibration generator includes an injector comprising an injector outer tube fixed inside a cylinder and an injector inner tube inserted into said injector outer tube, and a piston which is provided slidably inside said cylinder and is driven by a crank coupled to a rotating disk to reciprocate. In said external vibration generator, said injector inner tube is not fixed to said piston and a distance between said injector outer tube and said piston is arbitrarily adjustable. Therefore, a forward motion of said piston allows said injector inner tube to be pushed to thereby drive out a filling solution in said injector into said solution transport path, causing the balloon to dilate, while said balloon contracts in conjunction with a backward motion of said piston, causing a filling solution in said solution transport path to flow into said injector. Besides, the amplitude and cycle of said vibrational wave are arbitrarily adjustable depending on a bore diameter of said injector and a rotating speed of said rotating disk. Further, driving duration of the filling solution is also arbitrarily adjustable depending on a distance between said injector outer tube and said piston.

A third aspect of the present invention is a radiofrequency thermal balloon catheter system comprising: a catheter shaft comprising an outer tube and an inner tube which are slidable to each other; a balloon provided between a distal end of said outer tube and a vicinity of a distal end of said inner tube; an electrode for delivery of radiofrequency current provided in a central portion of said balloon, an external radiofrequency generator which supplies radiofrequency energy to said electrode for delivery of radiofrequency current; a temperature sensor provided inside said balloon, an external thermometer which detects temperature of said temperature sensor; a solution transport path which is formed between said outer tube and said inner tube, in communication with an inside of said balloon; an external vibration generator which applies vibrational waves to the inside of said balloon through said solution transport path; and a guide wire which guides said balloon to a target site, wherein said external vibration generator includes an injector comprising an injector outer tube fixed to an inside of a cylinder and an injector inner tube inserted into the injector outer tube, and a diaphragm which is fixed inside said cylinder and is driven by a crank coupled to a rotating disk to reciprocate. In said external vibration generator, said injector inner tube is fixed to said diaphragm and therefore a forward motion of said diaphragm allows said injector inner tube to be pushed to thereby drive out a filling solution in said injector into said solution transport path, causing said balloon to dilate, while a backward motion of said diaphragm allows said injector inner tube to be pulled to draw a filling solution in said solution transport path into said injector, causing said balloon to contract. Besides, the amplitude and cycle of said vibrational wave are arbitrarily adjustable depending on a bore diameter of said injector and a rotating speed of said rotating disk.

A fourth aspect of the present invention is a radiofrequency thermal balloon catheter system comprising: a catheter shaft comprising an outer tube and an inner tube which are slidable to each other; a balloon provided between a distal end of said outer tube and a vicinity of a distal end of said inner tube; an electrode for delivery of radiofrequency current provided in a central portion of said balloon, an external radiofrequency generator which supplies radiofrequency energy to said electrode for delivery of radiofrequency current; a temperature sensor provided inside said balloon, an external thermometer which detects temperature of said temperature sensor; a solution transport path which is formed between said outer tube and said inner tube, in communication with an inside of said balloon; an external vibration generator which applies vibrational waves to the inside of said balloon through said solution transport path; and a guide wire which guides said balloon to a target site, wherein said external vibration generator includes an injector comprising an injector outer tube fixed to an inside of a cylinder and an injector inner tube inserted into the injector outer tube, and a diaphragm which is fixed inside said cylinder and is driven by a crank coupled to a rotating disk to reciprocate. In said external vibration generator, said injector inner tube is not fixed to said diaphragm and a distance between said injector outer tube and said diaphragm is adjustable. Therefore, a forward motion of said diaphragm allows said injector inner tube to be pushed to drive out a filling solution in said injector into said solution transport path, causing the balloon to dilate, while said balloon contracts in conjunction with a backward motion of said diaphragm, causing a filling solution in said solution transport path to flow into said injector. Besides, the amplitude and cycle of said vibrational wave are arbitrarily adjustable depending on a bore diameter of said injector and a rotating speed of said rotating disk. Further, driving duration of the filling solution in said injector is also arbitrarily adjustable depending on a distance between said injector outer tube and said diaphragm.

A fifth aspect of the present invention is a radiofrequency thermal balloon catheter system comprising: a catheter shaft comprising an outer tube and an inner tube which are slidable to each other; a balloon provided between a distal end of said outer tube and a vicinity of a distal end of said inner tube; an electrode for delivery of radiofrequency current provided in a central portion of said balloon, an external radiofrequency generator which supplies radiofrequency energy to said electrode for delivery of radiofrequency current; a temperature sensor provided inside said balloon, an external thermometer which detects temperature of said temperature sensor; a solution transport path which is formed between said outer tube and said inner tube, in communication with an inside of said balloon; an external vibration generator which applies vibrational waves to the inside of said balloon through said solution transport path; and a guide wire which guides said balloon to a target site, wherein said external vibration generator includes an injector comprising an injector outer tube fixed inside a cylinder and an injector inner tube inserted into said injector outer tube, and a piston-shaped permanent magnet which is slidably provided inside said cylinder and is driven by magnetic force periodically generated by an electromagnet to reciprocate. In said external vibration generator, said injector inner tube is fixed to said piston-shaped permanent magnet and therefore the amplitude and cycle of said vibrational wave and driving duration of a filling solution in said injector are arbitrarily adjustable depending on a magnitude, cycle and duration of a current applied to said electromagnet.

A sixth aspect of the present invention is a radiofrequency thermal balloon catheter system comprising: a catheter shaft comprising an outer tube and an inner tube which are slidable to each other; a balloon provided between a distal end of said outer tube and a vicinity of a distal end of said inner tube; an electrode for delivery of radiofrequency current provided in a central portion of said balloon, an external radiofrequency generator which supplies radiofrequency energy to said electrode for delivery of radiofrequency current; a temperature sensor provided inside said balloon, an external thermometer which detects temperature of said temperature sensor; a solution transport path which is formed between said outer tube and said inner tube, in communication with an inside of said balloon; an external vibration generator which applies vibrational waves to the inside of said balloon through said solution transport path; and a guide wire which guides said balloon to a target site, wherein said external vibration generator includes an injector comprising an injector outer tube fixed inside a cylinder and an injector inner tube inserted into said injector outer tube, and a piston-shaped permanent magnet which is slidably provided inside said cylinder and is driven by magnetic force periodically generated by an electromagnet to reciprocate In said external vibration generator, a distance between the injector outer tube and said piston-shaped magnet is adjustable and therefore the amplitude and cycle of said vibrational wave and driving duration of a filling solution in said injector are arbitrarily adjustable depending on a magnitude, cycle and duration of a current applied to said electromagnet.

According to the radiofrequency thermal balloon catheter system of the present invention, the external vibration generator is equipped with the injector and the piston driven by the crank coupled to the rotating disk to reciprocate. Then, the amplitude of the vibrational wave is arbitrarily settable by reciprocating the injector inner tube by virtue of the piston to adjust the amplitude and cycle of the vibrational wave depending on the bore diameter of the injector and the rotating speed of the rotating disk. Hence, a radiofrequency thermal balloon catheter system can be provided which is equipped with a vibration generator that is not easily damaged and can generate powerful vibrational waves.

Further, the external vibration generator is equipped with the injector and the diaphragm driven by the crank coupled to the rotating disk to reciprocate. Then, the amplitude of the vibrational wave is arbitrarily settable by reciprocating the injector inner tube by virtue of the diaphragm to adjust the amplitude and cycle of the vibrational wave depending on the bore diameter of the injector and the rotating speed of the rotating disk. Hence, a radiofrequency thermal balloon catheter system can be provided which is equipped with a vibration generator that is not easily damaged and can generate the powerful vibrational waves.

Furthermore, the external vibration generator is equipped with the injector and the piston-shaped permanent magnet driven by magnetic force of the electromagnet to reciprocate. Then, the amplitude of the vibrational wave is arbitrarily settable by adjusting the amplitude and cycle of the vibrational wave and the driving duration of the filling solution in the injector depending on the magnitude, cycle and duration of the current applied to the electromagnet. Hence, a radiofrequency thermal balloon catheter system can be provided which is equipped with a vibration generator that is not easily damaged and can generate the powerful vibrational waves.

### BRIEF DESCRIPTION OF THE DRAWING

FIG. 1 is a schematic view illustrating an overall structure of a radiofrequency thermal balloon catheter system according to a first embodiment of the present invention.

FIG. 2 is a schematic view illustrating an external vibration generator of the radiofrequency thermal balloon catheter system according to the first embodiment of the present invention.

FIGS. 3(A) and 3(B) are schematic views illustrating behavior of the external vibration generator of the radiofrequency thermal balloon catheter system according to the first embodiment of the present invention.

FIG. 4 is a graph illustrating a cyclic change in amplitude of an external vibrational wave in the radiofrequency thermal balloon catheter system according to the first embodiment of the present invention.

FIG. 5 is a schematic view illustrating an external vibration generator in a radiofrequency thermal balloon catheter system according to a second embodiment of the present invention.

FIG. 6 is a schematic view illustrating an external vibration generator in a radiofrequency thermal balloon catheter system according to a third embodiment of the present invention.

FIG. 7 is another schematic view illustrating the external vibration generator of the radiofrequency thermal balloon catheter system according to the third embodiment of the present invention.

FIG. 8 is a schematic view illustrating how the radiofrequency thermal balloon catheter system according to the present invention is actually used.

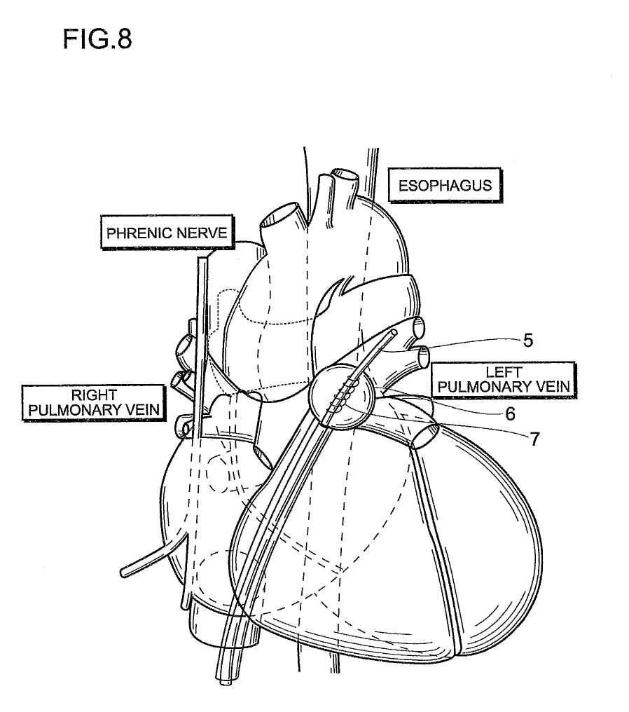

### DETAILED DESCRIPTION OF THE PREFERRED EMBODIMENTS

As follows is a detailed description of embodiments of a radiofrequency thermal balloon catheter system according to the present invention with reference to the appended drawings.

### Embodiment 1

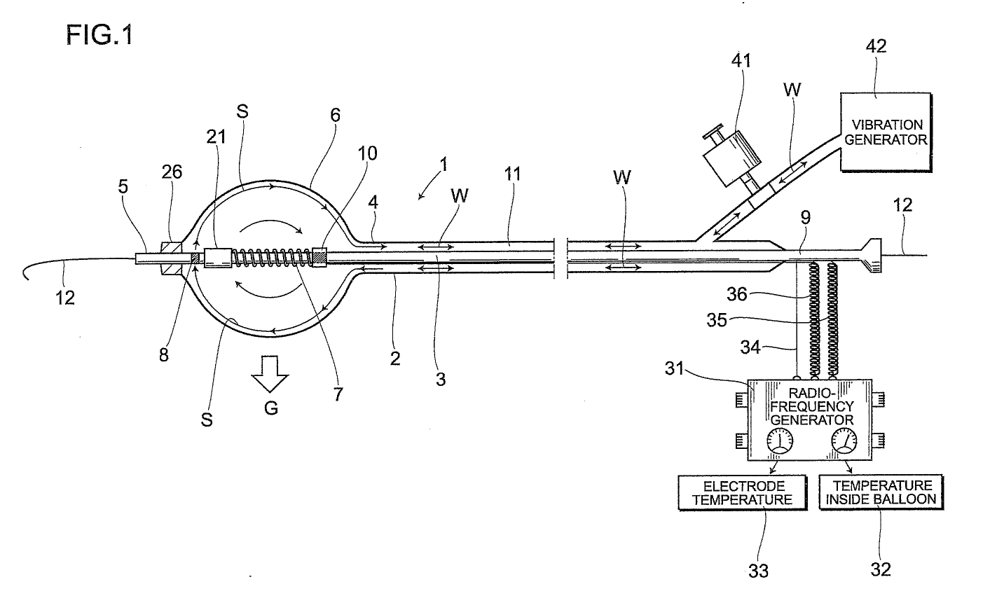

In FIGS. 1 to 3 showing a radiofrequency thermal balloon catheter system according to a first embodiment, numeral symbol 1 denotes a catheter shaft, which comprises an outer tube 2 and an inner tube 3 which are slidable to each other. A balloon 6 is provided between a distal end 4 of the outer tube 2 and a vicinity of a distal end 5 of the inner tube 3. The balloon 6 is formed from synthetic resin such as polyurethane or the like. The balloon 6 dilates into an approximately spherical form by filling an inside of the balloon 6 with a solution.

Inside the balloon 6, an electrode 7 for delivery of radiofrequency current for heating the inside of the balloon 6 is wound around the inner tube 3 in a coiled fashion to be provided in a central part of the balloon 6. The electrode 7 for delivery of radiofrequency current is monopolar and is able to conduct a radiofrequency current between itself and a counter electrode plate, not shown, provided outside the catheter shaft 1. Then, the electrode 7 for delivery of radiofrequency current generates heat by applying the radiofrequency current thereto. Alternatively, the electrode 7 for delivery of radiofrequency current may be bipolar to apply a radiofrequency current across both electrodes.

Further, inside the balloon 6, a temperature sensor 8 for detecting the temperature inside the balloon 6 is fixed in the vicinity of the distal end 5 of the inner tube 3. Besides, an electrode temperature sensor 10 for detecting the temperature of the electrode 7 for delivery of radiofrequency current is arranged in contact with the electrode 7 for delivery of radiofrequency current, and is fixed on a side closer to a proximal end 9 of the inner tube 3.

Between the outer tube 2 and the inner tube 3, a solution transport path 11 is formed which communicates with the inside of the balloon 6. A guide wire 12 for guiding the balloon 6 to a target site is provided in such a fashion as to be inserted through the inner tube 3.

Between the temperature sensor 8 and the electrode 7 for delivery of radiofrequency current, a heat insulating material 21 is interposed. Thus, the temperature sensor 8 can be prevented from being directly heated by the electrode 7 for delivery of radiofrequency current and thus the temperature inside the balloon 6 can be precisely detected.

In the vicinity of the distal end 5 of the inner tube 3, a balloon external heat shield knob 26 is provided in contact with an outer surface of the balloon 6. As a result, the temperature sensor 8 provided in the vicinity of the distal end 5 of the inner tube 3 inside the balloon 6 can be prevented from being affected by temperature of blood or the like contacting the balloon 6, thus permitting the temperature inside the balloon 6 to be precisely detected. In addition, the inner tube 3 penetrates through a central portion of the balloon external heat shield knob 26, which is fixed to the inner tube 3.

Outside the catheter shaft 1, there are provided an external radiofrequency generator 31 for supplying radiofrequency energy for heating the balloon 6 to the electrode 7 for delivery of radiofrequency current, an external thermometer 32 for indicating the temperature detected by the temperature sensor 8, and an external electrode thermometer 33 for indicating the temperature detected by the electrode temperature sensor 10. The electrode 7 for delivery of radiofrequency current and the external radiofrequency generator 31 are connected electrically to each other through a lead wire 34, while the temperature sensor 8 and the external thermometer 32, the electrode temperature sensor 10 and the external electrode thermometer 33, are connected electrically to each other by lead wires 35, 36, respectively. Further, between the distal end 5 of the inner tube 3 and the proximal end 9 thereof, the lead wires 34, 35 and 36 are fixed to the inner tube 3.

Furthermore, outside the catheter shaft 1, there are provided a syringe 41 for supplying the solution to the balloon 6 through the solution transport path 11 and an external vibration generator 42 for applying asymmetric vibrational waves to the balloon 6 through the solution transport path 11 to steadily generate swirls S inside the balloon 6. Then, a diameter of the balloon 6 is changed by varying pressure of the solution supplied to the balloon 6 by means of the syringe 41. The solution inside the balloon 6 is agitated by the swirls S to keep the temperature inside the balloon 6 uniform.

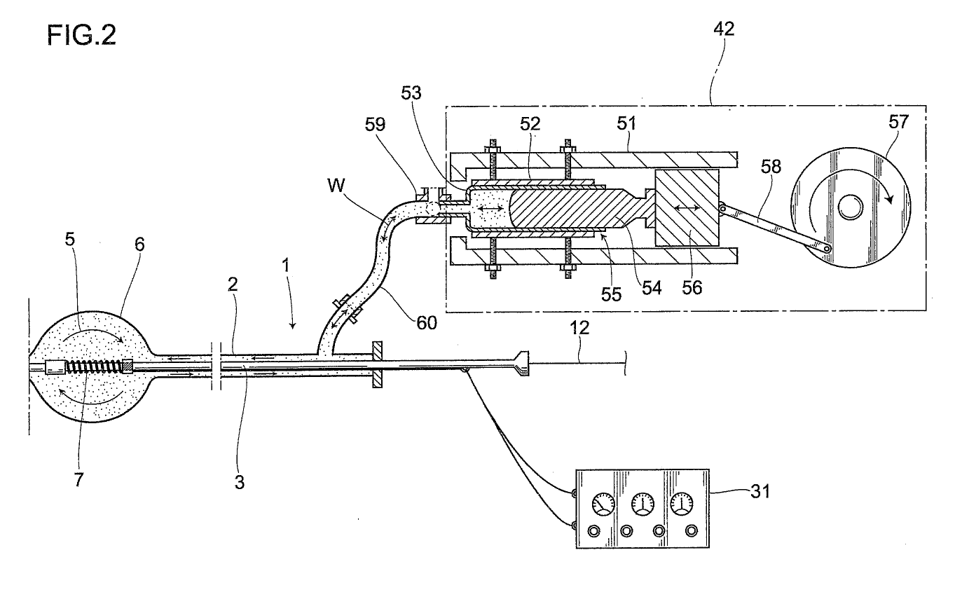

In FIG. 2 showing details of the external vibration generator 42, the external vibration generator 42 is equipped with a cylinder 51, inside which an injector outer tube 53 is fixed with a fixing unit 52. An injector inner tube 54 is inserted into an injector outer tube 53 and both the inner and outer tubes make up an injector 55. Inside the cylinder 51, a piston 56 is slidably provided and is driven by a crank 58 coupled to a rotating disk 57. Further, a distal end of the injector 55 is coupled to the catheter shaft 1 through a three-way cock 59 and a coupling tube 60.

Here, the injector inner tube 54 may be fixed to the piston 56 or may not be fixed thereto.

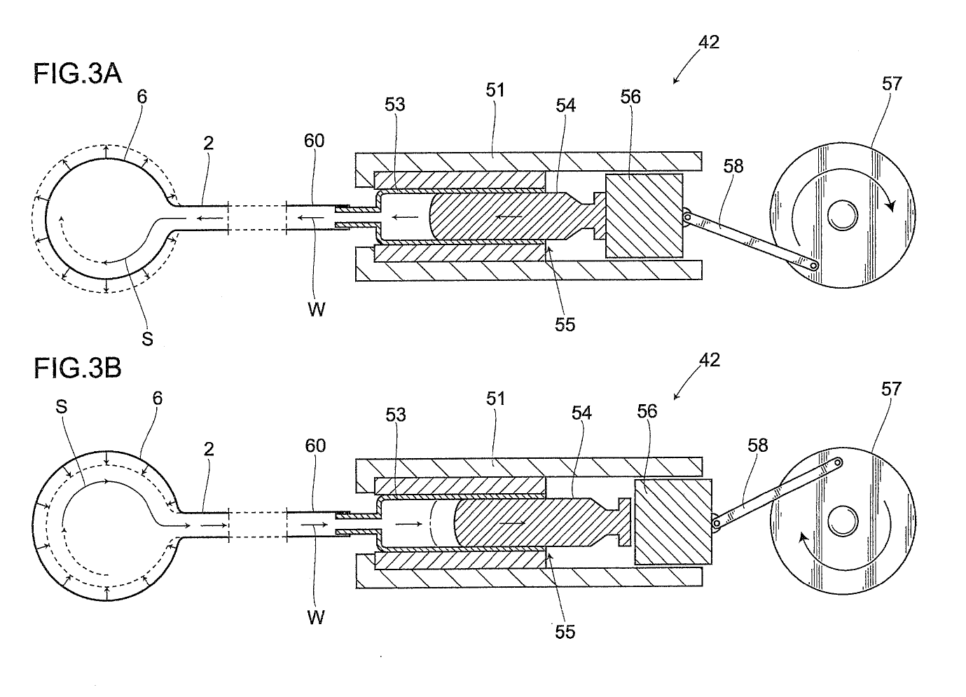

When the injector inner tube 54 is fixed to the piston 56, as shown in FIG. 3(A), a forward motion of the piston 56 allows the injector inner tube 54 to be pushed to thereby drive out a filling solution in the injector 55 into the solution transport path 11, causing the balloon 6 to dilate, while a backward motion of the piston 56 allows the injector inner tube 54 to be pulled to thereby draw a filling solution in the solution transport path 11 into the injector 55, causing the balloon 6 to contract. In other words, when the backward motion of the piston 56 starts, the backward motion of the piston 56 brings negative pressure into an inside of the balloon 6 and therefore drawing force is applied to the solutions inside the solution transport path 11 and inside the balloon 6. Therefore, the reciprocating motion of the piston 56 generates vibrational waves W.

In contrast, when the injector inner tube 54 is not fixed to the piston 56, a distance between the injector outer tube 53 and the piston 56 is so structured as to be adjustable. As shown in FIG. 3(B), a forward motion of the piston 56 allows the injector inner tube 54 to be pushed to thereby drive out the filling solution in the injector 55 into the solution transport 11, causing the balloon 6 to dilate. Contrarily, the balloon 6 contracts in conjunction with the backward motion of the piston 56, causing the filling solution in the solution transport path 11 to flow into the injector 55. Specifically, when the backward motion of the piston 56 starts, the balloon 6 contracts to feed the filling solution into the injector 55 through the solution transport path 11. At this time, the filling solution moves only by deflating force of the balloon 6. Then, this reciprocating motion of the piston 56 generates the vibrational waves W.

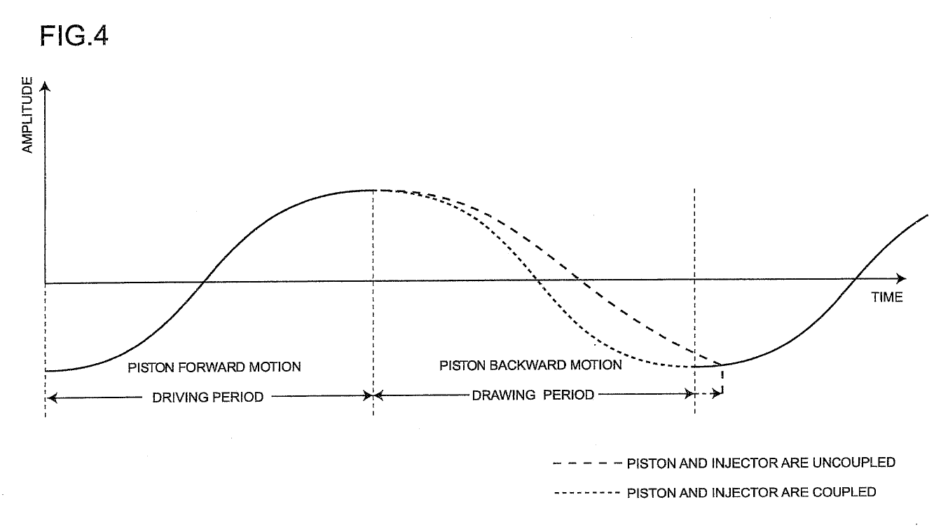

Cyclic changes of amplitudes of the vibrational waves W are shown in FIG. 4 when the injector inner tube 54 is coupled to the piston 56 and when the injector inner tube 54 is uncoupled from the piston 56. The filling solution in the injector 55 is driven out to the inside of the balloon 6 by the forward motion of the piston 56, resulting in a significantly rising curve drawn. Subsequently, the filling solution inside the balloon 6 is drawn by the backward motion of the piston 56. In addition, in each of states where the injector inner tube 54 and the piston 56 are coupled and uncoupled, the vibrational waves are generated but in different waveforms. In the coupled state, a symmetric waveform of the vibrational wave is generated, while in the uncoupled state, an asymmetric one is generated.

In addition, amplitude of the vibrational wave W is arbitrarily adjustable by varying a diameter of a tube of the injector 55, i.e., its bore diameter. A cycle of the vibrational wave W is arbitrarily adjustable by varying a rotating speed of the rotating disk 57. Further when the injector inner tube 54 is uncoupled from the piston 56, driving duration of the filling solution in the injector 55 is arbitrarily adjustable by varying the distance between the injector outer tube 53 and the piston 56.

Next is a description of how to use the radiofrequency thermal balloon catheter system according to the present embodiment.

First, liquids such as physiologic saline, a contrast agent or the like are infused from the syringe 41 into the insides of the solution transport path 11 and balloon 6 to thereby perform air bleeding. Then, the balloon 6 is allowed to contract with the outer and inner tubes mutually slid so as to maximize a distance between the distal end 4 of the outer tube 3 and that 5 of the inner tube 3.

Then, by the aid of the guide wire 12, a sheath-shaped guiding sheath for guiding the catheter shaft 1 is inserted into a vicinity of a target site inside a patient body. The contracted balloon 6 is inserted into the guiding sheath to make the balloon 6 stay in the vicinity of the target site.

Next, the solution is infused from the syringe 41 into the balloon 6 to dilate the balloon 6. Here, the balloon 6 is adjusted in length by adjusting the distance between the distal end 4 of the outer tube 2 and the distal end 5 of the inner tube 3 and then the balloon 6 is adjusted in diameter by adjusting pressure of the solution supplied to the balloon 6 by the syringe 41. Then, the balloon 6 is pressed against the target site.

Subsequently, the lead wires 34, 35 and 36 connected to the electrode 7 for delivery of radiofrequency current, the temperature sensor 8 and the electrode temperature sensor 10, respectively, are connected, from the basal portion 9 of the inner tube 3, with the radiofrequency generator 31, the thermometer 32 and the electrode thermometer 33, respectively. Then, an output of the radiofrequency generator 31 is built up while observing the thermometer 32 and the electrode thermometer 33. When starting the vibration generator 42, the rotating disk 57 begins to rotate and then the piston 56 starts reciprocating by the crank 58 to move back and forth the injector inner tube 54 in contact with the piston 56, thus generating the vibrational waves W. Due to the vibrational waves W, the inside of the balloon 6 is agitated to make the temperature of the balloon 6 uniform. Then, the target site in contact with the balloon 6 is ablated while adjusting the surface temperature of the balloon 6 and the current conducting duration.

As described above, the radiofrequency thermal balloon catheter system according to the present embodiment is equipped with the catheter shaft 1 comprising the outer tube 2 and the inner tube 3 which are slidable to each other; the balloon 6 provided between the distal end 4 of the outer tube 2 and the vicinity of the distal end 5 of the inner tube 3; the electrode 7 for delivery of radiofrequency current provided in the central portion of the balloon 6; the external radiofrequency generator 31 which supplies radiofrequency energy to the electrode 7 for delivery of radiofrequency current; the temperature sensor 8 provided inside the balloon 6; the external thermometer 32 which detects the temperature of the temperature sensor 8; the solution transport path 11 formed between the outer tube 2 and the inner tube 3, in communication with the inside of the balloon 6; the external vibration generator 42 which applies the vibrational waves W to the inside of the balloon 6 through the solution transport path 11; and the guide wire 12 which guides the balloon 6 to a target site. In the radiofrequency thermal balloon catheter system thus structured, the external vibration generator 42 is equipped with the injector 55 comprising the injector outer tube 53 fixed inside the cylinder 51 and the injector inner tube 54 inserted into the injector outer tube 53, and the piston 56 which is slidably provided inside the cylinder 51 and is driven by the crank 58 coupled to the rotating disk 57 to reciprocate. Further, the injector inner tube 54 is fixed to the piston 56 and therefore the forward motion of the piston 56 allows the injector inner tube 54 to be pushed to thereby drive out a filling solution in the injector 55 into the solution transport path 11, causing the balloon 6 to dilate, while the backward motion of the piston 56 allows the injector inner tube 54 to be pulled to thereby draw a filling solution in the solution transport path 11 into the injector 55, causing the balloon 6 to contract. Besides, the amplitude and cycle of the vibrational wave W are arbitrarily adjustable depending on the bore diameter of the injector 55 and the rotating speed of the rotating disk 57.

Otherwise, the radiofrequency thermal balloon catheter system in the present embodiment is equipped with the catheter shaft 1 comprising the outer tube 2 and the inner tube 3 which are slidable to each other; the balloon 6 provided between the distal end 4 of the outer tube 2 and the vicinity of the distal end 5 of the inner tube 3; the electrode 7 for delivery of radiofrequency current provided in the central portion of the balloon 6; the external radiofrequency generator 31 which supplies radiofrequency energy to the electrode 7 for delivery of radiofrequency current; the temperature sensor 8 provided inside said balloon 6; the external thermometer 32 which detects the temperature of the temperature sensor 8; the solution transport path 11 formed between the outer tube 2 and the inner tube 3, in communication with the inside of the balloon 6; the external vibration generator 42 which applies vibrational waves to the inside of the balloon 6 through the solution transport path 11; and the guide wire 12 which guides the balloon 6 to a target site. In the radiofrequency thermal balloon catheter system thus structured, the external vibration generator 42 is equipped with the injector 55 comprising the injector outer tube 53 fixed inside the cylinder 51 and the injector inner tube 54 inserted into the injector outer tube 53, and the piston 56 which is provided slidably inside the cylinder 51 and is driven by the crank 58 coupled to the rotating disk 57 to reciprocate. Further, the injector inner tube 54 is not fixed to the piston 56 and the distance between the injector inner tube 54 and the piston 56 is adjustable. Therefore, the forward motion of the piston 56 allows the injector inner tube 54 to be pushed to thereby drive out a filling solution in the injector into the solution transport path 11, causing the balloon 6 to dilate, while the balloon 6 contracts in conjunction with the backward motion of the piston 56, causing a filling solution in the solution transport path 11 to flow into the injector 55. Besides, the amplitude and cycle of the vibrational wave W are arbitrarily adjustable depending on the bore diameter of the injector 55 and the rotating speed of the rotating disk 57. Further, the driving duration of the filling solution is also arbitrarily adjustable depending on the distance between the injector outer tube 53 and the piston 56.

According to the radiofrequency thermal balloon catheter system in the first embodiment, the external vibration generator 42 is equipped with the injector 55 and the piston 56 driven by the crank 58 coupled to the rotating disk 57 to reciprocate. Then, the amplitude of the vibrational wave W is arbitrarily settable by reciprocating the injector inner tube 54 by virtue of the piston 56 to adjust the amplitude and cycle of the vibrational wave W depending on the bore diameter of the injector 55 and the rotating speed of the rotating disk 57. Hence, the radiofrequency thermal balloon catheter system can be provided which is equipped with the vibration generator that is not easily damaged and can generate powerful vibrational waves W.

Further, by employing a new and expendable sterilized injector tube as the injector 55, it is less likely to be damaged, and even if damaged, the injector can be simply replaced. Besides, if an injector tube different in diameter is employed, the vibrational wave W can be easily changed in amplitude. Furthermore, by coupling the injector inner tube 54 to the piston 56, not only a driving motion but a drawing motion can be practiced by strong external force. Hence, smooth and powerful vibrational waves can be generated.

### Embodiment 2

A radiofrequency thermal balloon catheter system according to a second embodiment is different only in the system of the external vibration generator 42 from that in the first embodiment. Specifically, the reciprocating motion of the piston is replaced by that of a diaphragm and therefore the principle of the system is the same as that in the first embodiment. Hereunder, the same numeral symbols are used for parts the same as those in the first embodiment and a detailed description thereof is omitted.

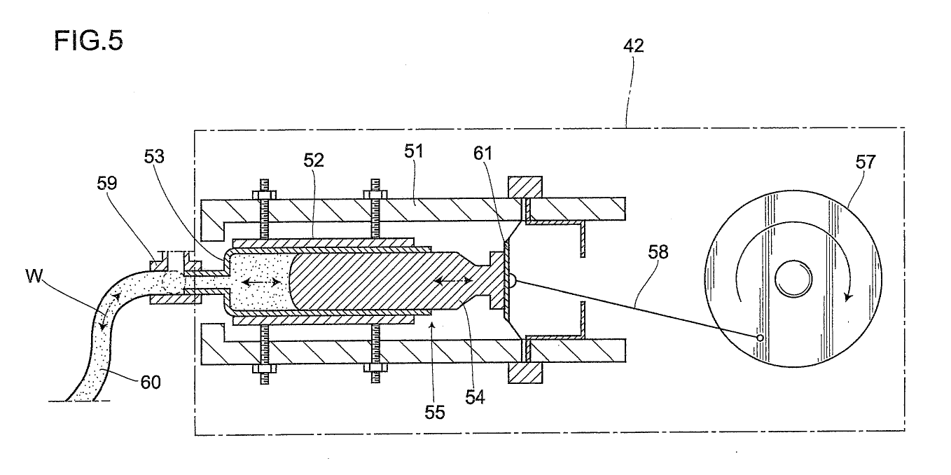

In FIG. 5 showing details of the external vibration generator 42 in the radiofrequency thermal balloon catheter system according to the second embodiment, the external vibration generator 42 is equipped with the cylinder 51, inside which the injector outer tube 53 is fixed by the fixing unit 52. The injector 55 comprises the injector outer tube 53 and the injector inner tubes 54 inserted into the injector outer tube 53. Besides, the diaphragm 61 is fixed inside the cylinder 51 and is driven by the crank 58 coupled to the rotating disk 57. The distal end of the injector 55 is coupled to the catheter shaft 1 through the three-way cock 59 and the coupling pipe 60.

Here, the injector inner tube 54 may be fixed to the diaphragm 61 or may not be fixed thereto.

Then, when the injector inner tube 54 is fixed to the diaphragm 61, a forward motion of the diaphragm 61 allows the injector inner tube 54 to be pushed to thereby drive out the filling solution in the injector 55 into the solution transport path 11, causing the balloon 6 to dilate, while a backward motion of the diaphragm 61 allows the injector inner tube 54 to be pulled to thereby draw the filling solution in the solution transport path 11 into the injector 55, causing the balloon 6 to contract. In other word, when the backward motion of the diaphragm 61 starts, the backward motion of the diaphragm 61 brings negative pressure into the inside of the injector 55 to apply force for drawing the filling solutions inside the solution transport path 11 and inside the balloon 6. As a result, a reciprocating motion of the diaphragm 61 generates the vibrational waves W.

In contrast, when the injector inner tube 54 is not fixed to the diaphragm 61, a distance between the injector outer tube 53 and the diaphragm 61 is so structured as to be adjustable. Therefore, the forward motion of the diaphragm 61 allows the injector inner tube 54 to be pushed to thereby drive out the filling solution in the injector 55 into the solution transport path 11, causing the balloon 6 to dilate, while the balloon 6 contracts in conjunction with the backward motion of said piston 56, causing a filling solution in said solution transport path to flow into the injector 55. In other word, when the backward motion of the diaphragm 61 starts, the balloon 6 contracts to feed the filling solution into the injector 55 through the solution transport path 11. At this time, the filling solution moves only by deflating force of the balloon 6. Then, the reciprocating motion of the balloon 6 generates the vibrational waves W.

In addition, the amplitude of the vibrational wave W is arbitrarily adjustable by varying the diameter of the tube of the injector 55, i.e., its bore diameter. The cycle of the vibrational wave W is arbitrarily adjustable by varying the rotating speed of the rotating disk 57. Further, when the injector inner tube 54 is uncoupled from the diaphragm 61, the driving duration of the filling solution in the injector 55 is arbitrarily adjustable depending on the distance between the injector outer tube 53 and the diaphragm 61.

As described above, the radiofrequency thermal balloon catheter system in the present embodiment is equipped with the catheter shaft 1 comprising the outer tube 2 and the inner tube 3 which are slidable to each other; the balloon 6 provided between the distal end 4 of the outer tube 2 and the vicinity of the distal end 5 of the inner tube 3; the electrode 7 for delivery of radiofrequency current provided in the central portion of the balloon 6; the external radiofrequency generator 31 which supplies radiofrequency energy to the electrode 7 for delivery of radiofrequency current; the temperature sensor 8 provided inside said balloon 6; the external thermometer 32 which detects the temperature of the temperature sensor 8; the solution transport path 11 formed between the outer tube 2 and the inner tube 3, in communication with the inside of the balloon 6; the external vibration generator 42 which applies vibrational waves to the inside of the balloon 6 through the solution transport path 11; and the guide wire 12 which guides the balloon 6 to a target site. In the radiofrequency thermal balloon catheter system thus structured, the external vibration generator 42 is equipped with the injector 55 comprising the injector outer tube 53 fixed inside the cylinder 51 and the injector inner tube 54 inserted into the injector outer tube 53, and the diaphragm 61 which is fixed inside the cylinder 51 and is driven by the crank 58 coupled to the rotating disk 57 to reciprocates. Further, the injector inner tube 54 is fixed to the diaphragm 61 and therefore the forward motion of the diaphragm 61 allows the injector inner tube 54 to be pushed to thereby drive out a filling solution in the injector 55 into the solution transport path 11, causing the balloon 6 to dilate, while the backward motion of the diaphragm 61 allows the injector inner tube 54 to be pulled to thereby draw the filling solution in the solution transport path 11 into the injector 55, causing the balloon 6 to contract. Besides, the amplitude and cycle of the vibrational wave are arbitrarily adjustable depending on the bore diameter of the injector 55 and the rotating speed of the rotating disk 57.

Otherwise, the radiofrequency thermal balloon catheter system in the present embodiment is equipped with the catheter shaft 1 comprising the outer tube 2 and the inner tube 3 which are slidable to each other; the balloon 6 provided between the distal end 4 of the outer tube 2 and the vicinity of the distal end 5 of the inner tube 3; an electrode for delivery of radiofrequency current 7 provided in the central portion of the balloon 6; the external radiofrequency generator 31 which supplies radiofrequency energy to the electrode 7 for delivery of radiofrequency current; the temperature sensor 8 provided inside said balloon 6, the external thermometer 32 which detects the temperature of the temperature sensor 8; the solution transport path 11 which is formed between the outer tube 2 and the inner tube 3, in communication with the inside of the balloon 6; the external vibration generator 42 which applies vibrational waves to the inside of the balloon 6 through said solution transport path 11; and the guide wire 12 which guides the balloon 6 to a target site. In the radiofrequency thermal balloon catheter system thus structured, the external vibration generator 42 is equipped with the injector 55 comprising the injector outer tube 53 fixed inside the cylinder 51 and the injector inner tube 54 inserted into the injector outer tube 53, and the diaphragm 61 which is fixed to the inside of the cylinder 51 and is driven by the crank 58 coupled to the rotating disk 57 to reciprocate. Further, the injector inner tube 54 is not fixed to the diaphragm 61 and the distance between the injector outer tube 53 and the diaphragm 61 is adjustable. Therefore, the forward motion of the diaphragm 61 allows the injector inner tube 54 to be pushed to thereby drive out the filling solution in the injector 55 into the solution transport path 11, causing the balloon 6 to dilate, while the balloon 6 contracts in conjunction with the backward motion of the diaphragm 61, causing the filling solution in the solution transport path 11 to flow into the injector 55. Besides, the amplitude and cycle of the vibrational wave W are arbitrarily adjustable depending on the bore diameter of the injector 55 and the rotating speed of the rotating disk 57. Furthermore, the driving duration of the filling solution in the injector 55 is also arbitrarily adjustable depending on the distance between the injector outer tube 53 and the diaphragm 61.

According to the radiofrequency thermal balloon catheter system in the second embodiment, the external vibration generator 42 is equipped with the injector 55 and the diaphragm 61 driven by the crank 58 coupled to the rotating disk 57 to reciprocate. Then, the amplitude of the vibrational wave W is arbitrarily settable by reciprocating the injector inner tube 54 by virtue of the diaphragm 61 to adjust the amplitude and cycle of the vibrational wave W depending on the bore diameter of the injector 55 and the rotating speed of the rotating disk 57. Hence, a radiofrequency thermal balloon catheter system can be provided which is equipped with the vibration generator that is not easily damaged and can generate powerful vibrational waves.

### Embodiment 3

A radiofrequency thermal balloon catheter system in a third present embodiment is different only in the system of the external vibration generator 42 from that in the first embodiment. Hereunder, the same numeral symbols are used for parts the same as those in the first embodiment and a detailed description thereof is omitted.

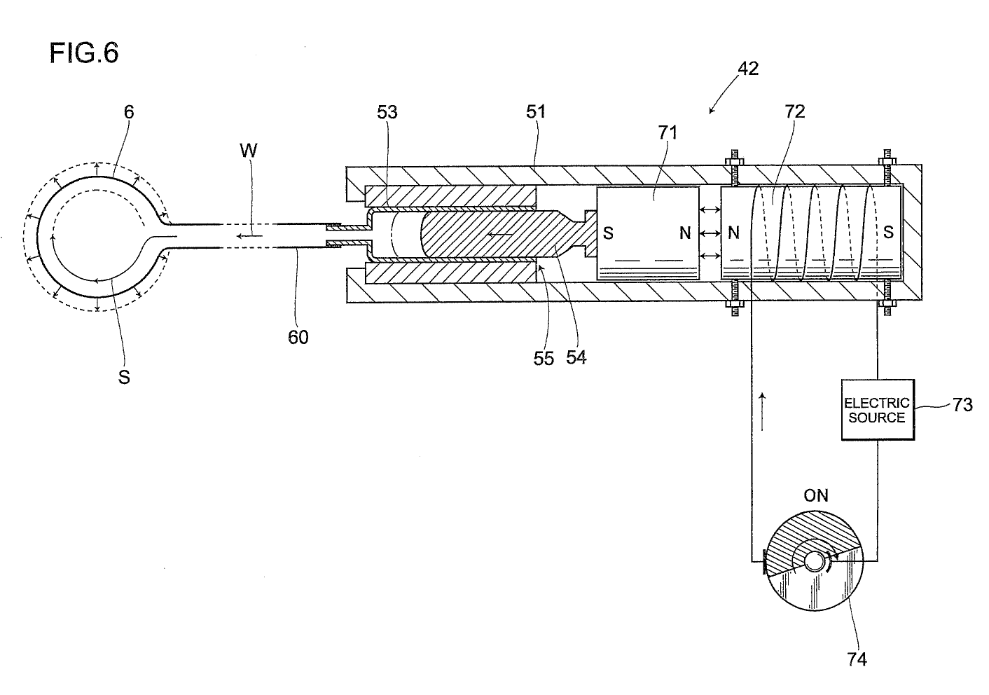

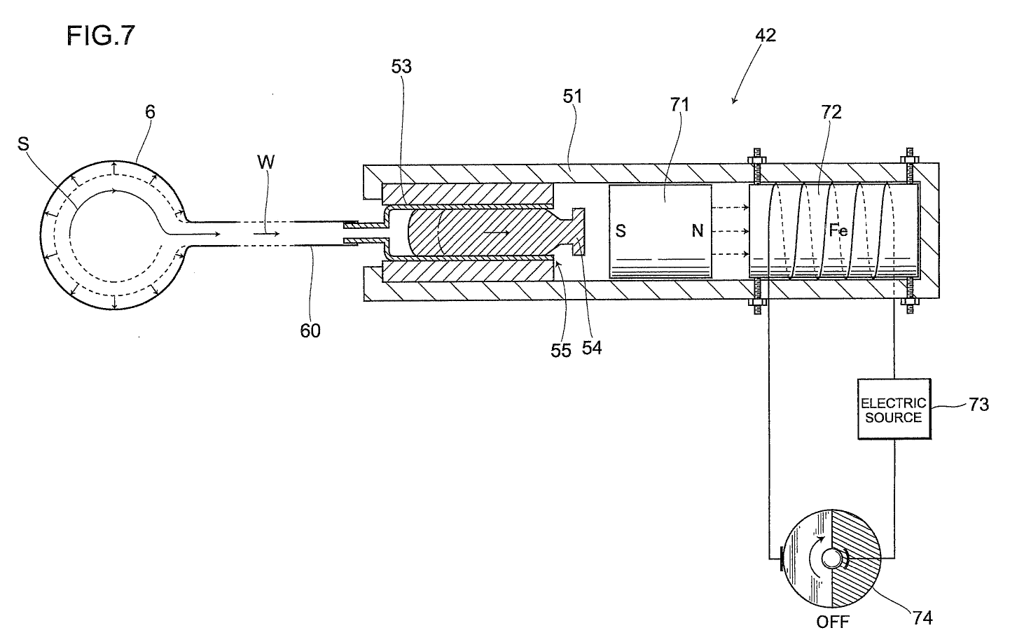

In FIGS. 6, 7 showing details of the external vibration generator 42 in the radiofrequency thermal balloon catheter system in the third embodiment, the external vibration generator 42 is equipped with a cylinder 51, inside which the injector outer tube 53 is fixed. The injector 55 comprises the injector outer tube 53 and the injector inner tube 54 inserted into the injector inner tube 53. Besides, a piston-shaped permanent magnet 71 is slidably provided inside the cylinder 51. Further, an electromagnet 72 is fixed inside the cylinder 51. The piston-shaped permanent magnet 71 is driven by magnetic force periodically generated by the electromagnet 72 to reciprocate. The electromagnet 72 is equipped with an electric source 73 and a switchboard 74 and then power supply from the electric source 73 can be turned on and off by the switchboard 74. Besides, the switchboard 74 is a rotary switch to be capable of performing on-off control of electric power applied to the electromagnet 72 at a constant frequency. When the electromagnet 72 is in a power-on state, the piston-shaped permanent magnet 71 repels against the electromagnet 72 to move forward.

Then, as shown in FIG. 6, when the electromagnet 72 has made the transition to a power-on state, the injector inner tube 54 is pushed by a forward motion of the piston-shaped permanent magnet 71 and thus the filling solution in the injector 55 is driven out into the solution transport 11 to dilate the balloon 6. Contrarily, as shown in FIG. 7, when the electromagnet 72 is turned to a power-off state, the electromagnet 72 becomes ineffective to change its own core into a mere iron bar. Hence, the piston-shaped permanent magnet 71 and the iron bar are attracted to each other to move the piston-shaped permanent magnet 71 backward. Then, the balloon 6 contracts by the backward motion of the piston-shaped permanent magnet 71 and as a result, the filling solution in the solution transport path 11 flows into injector 55. In other words, when the backward motion of the piston-shaped permanent magnet 71 starts, the balloon 6 contracts to feed the filling solution into the injector 55 through the solution transport path 11. At this time, the filling solution moves only by deflating force of the balloon 6. In addition, the injector inner tube 54 may be fixed to the piston-shaped permanent magnet 71 and in such a case the injector inner tube 54 is pulled by the backward motion of the piston-shaped permanent magnet 71 and thus the filling solution in the solution transport 11 is drawn by the injector 55 to contract the balloon 6. As a result, the piston-shaped permanent magnet 71 is reciprocated to generate the vibrational waves W.

In addition, the amplitude and cycle of the vibrational wave W and the driving duration of the filling solution in the injector 55 are arbitrarily adjustable depending on a magnitude, cycle and duration of a current applied to the electromagnet 72.

As described above, the radiofrequency thermal balloon catheter system in the present embodiment is equipped with the catheter shaft 1 comprising the outer tube 2 and the inner tube 3 which are slidable to each other; the balloon 6 provided between the distal end 4 of the outer tube 2 and the vicinity of the distal end 5 of the inner tube 3; the electrode 7 for delivery of radiofrequency current provided in the central portion of the balloon 6; the external radiofrequency generator 31 which supplies radiofrequency energy to the electrode 7 for delivery of radiofrequency current; the temperature sensor 8 provided inside said balloon 6, the external thermometer 32 which detects the temperature of the temperature sensor 8; the solution transport path 11 which is formed between the outer tube 2 and the inner tube 3, in communication with the inside of the balloon 6; the external vibration generator 42 which applies vibrational waves to the inside of the balloon 6 through said solution transport path 11; and the guide wire 12 which guides the balloon 6 to a target site. In the radiofrequency thermal balloon catheter system thus structured, the external vibration generator 42 is equipped with the injector 55 comprising the injector outer tube 53 fixed inside the cylinder 51 and the injector inner tube 54 inserted into the injector outer tube 53, and the piston-shaped permanent magnet 7 which is slidably provided inside the cylinder 51 and is driven by magnetic force periodically generated by the electromagnet 72 to reciprocate. Besides, the injector inner tube 54 is fixed to the piston-shaped permanent magnet 7 and therefore the amplitude and cycle of the vibrational wave W and the driving duration of the filling solution in the injector 55 are arbitrarily adjustable depending on the magnitude, cycle and duration of the current applied to the electromagnet 72.

Otherwise, the radiofrequency thermal balloon catheter system in the present embodiment is equipped with the catheter shaft 1 comprising the outer tube 2 and the inner tube 3 which are slidable to each other; the balloon 6 provided between the distal end 4 of the outer tube 2 and the vicinity of the distal end 5 of the inner tube 3; the electrode 7 for delivery of radiofrequency current provided in the central portion of the balloon 6; the external radiofrequency generator 31 which supplies radiofrequency energy to the electrode 7 for delivery of radiofrequency current; the temperature sensor 8 provided inside said balloon 6, the external thermometer 32 which detects the temperature of the temperature sensor 8; the solution transport path 11 which is formed between the outer tube 2 and the inner tube 3, in communication with the inside of the balloon 6; the external vibration generator 42 which applies vibrational waves to the inside of the balloon 6 through said solution transport path 11; and the guide wire 12 which guides the balloon 6 to a target site. In the radiofrequency thermal balloon catheter system thus structured, the external vibration generator 42 is equipped with the injector 55 comprising the injector outer tube 53 fixed inside the cylinder 51 and the injector inner tube 54 inserted into the injector outer tube 53, and the piston-shaped permanent magnet 7 which is provided slidably inside the cylinder 51 and is driven by magnetic force periodically generated by electromagnet 72 to reciprocate. Besides, a distance between the injector outer tube 53 and the piston-shaped permanent magnet 7 is adjustable. Therefore, the amplitude and cycle of the vibrational wave W and the driving duration of the filling solution in the injector 55 are arbitrarily adjustable depending on the magnitude, cycle and the duration of the current applied to the electromagnet 72.

According to the radiofrequency thermal balloon catheter system in the third embodiment, the external vibration generator 42 is equipped with the injector 55 and the piston-shaped permanent magnet 7 driven by the magnetic force of the electromagnet 72 to reciprocate. Then, the amplitude of the vibrational wave W is arbitrarily settable by adjusting the amplitude and cycle of the vibrational wave W and the driving duration of the filling solution in the injector 55 depending on the magnitude, cycle and duration of the current applied to the electromagnet 72. Hence, the radiofrequency thermal balloon catheter system can be provided which is equipped with the vibration generator that is not easily damaged and can generate powerful vibrational waves W.

### Embodiment 4

In the present embodiment, one example is shown in which the radiofrequency thermal balloon catheter system in the first embodiment of the present invention is applied to an ablative therapy of an atrial fibrillation (AF). The radiofrequency thermal balloon catheter system takes advantage of performing an area to area ablation, different from a point to point ablation by the conventional electrode catheter. Hence, not only pulmonary vein isolation but pulmonary vein antrum isolation and left atrial posterior wall isolation can be simultaneously performed in a short procedure time, thus improving the therapeutic effect. Extensive ablation, however, might cause collateral damage on adjacent organs such as phrenic nerve paralysis, esophageal ulcer or the like. With respect to this system, however, such damage can be prevented by using the external vibration generator 42 in the following manner to perform a uniform heating of the balloon.

As shown in FIG, 8, under general anesthesia, the radiofrequency thermal balloon catheter was inserted into a left atrium in a patient with a drug-resistant AF. Then, using the catheter system according to the present invention, an isolation of a left atrial posterior wall including all pulmonary veins was performed on the patient under artificial respiration. A mixed liquid containing a physiologic saline and a contrast medium was preliminarily filled into the balloon 6, the solution transport path 11, the coupling tube 60 and the injector 55. Thereafter, a proximal extremity of the injector inner tube 54 was set so as to come in contact with the piston 56 of the external vibration generator 42 with the injector 55 fixed inside a housing of the external vibration generator 42. Then, after inserting the balloon 6 from a femoral vein and imaging the entire pulmonary veins, a diameter and wall thickness of the pulmonary vein were measured using an ICE and then the balloon 6 was wedged into the antrum. The delivery time of radiofrequency current was determined depending on the wall thickness of the atrium (2 minutes for 2 mm wall thickness, 3 minutes for 3 mm wall thickness) and the central temperature of the balloon 6 was set between 65 degrees C. and 75 degrees C. depending on the diameter of the balloon 6. Then, the external vibration generator 42 was activated a with a radiofrequency current applied at an output of 50 W to 150 W. At this moment, the amplitude and cycle of the vibrational wave W were regulated so that swirls S were generated up-and-down against the gravity inside the balloon 6. By wedging the balloon into the antrum, the pulmonary veins were isolated and then while dragging the balloon 6, conduction block lines were formed at the roof between upper pulmonary veins and at the posterior wall between lower pulmonary veins.

Thus, after the above ablation therapy was applied to 100 cases, the follow-up research implemented for a year demonstrated that 94% paroxysmal AFs (63 cases) and 86% persistent AFs (37 cases) became medicament-free. Further, no damage to adjacent internal organs occurred.

In the radiofrequency thermal balloon catheter system, the radiofrequency current is not applied directly to an organ and the most of the applied energy is used to heat the filling solution inside the balloon 6. At the same time, when the amplitude and cycle of a vibrational wave are adjusted using the external vibration generator 42 and the swirls S against the gravity are generated inside the balloon 6, a temperature difference caused by convection is canceled to uniformly heat the entire balloon 6 and thus an organ in contact with the balloon 6 is uniformly ablated by heat conduction. At this moment, a gentle negative temperature gradient is established from the center of the balloon 6 to the deep tissue. A lesion depth caused by the heat conduction from the balloon 6 is proportional to a balloon contact temperature and a delivery time of radiofrequency current. With the balloon contact temperature set at 60 degrees C., the ablation depth is 2 mm at a 2 minutes' delivery time of radiofrequency current and is 3 mm at a 3 minutes' delivery time. Therefore, if a wall thickness of a target site is measured using an intracardiac echo to set the temperature and the delivery time of radiofrequency current in advance, only a target organ is ablated to bring about a cure, without collateral damage to adjacent internal organs.

An isolation of a left posterior atrial wall including all pulmonary veins by means of the radiofrequency thermal balloon catheter system utilizing the external radiofrequency generator 31 in conjunction with the external vibration generator 42 is a safe and useful treatment for AFs.

What is claimed is:

1. A radiofrequency thermal balloon catheter system comprising:
a catheter shaft comprising an outer tube and an inner tube which are slidable to each other;
a balloon provided between a distal end of said outer tube and a vicinity of a distal end of said inner tube;
an electrode for delivery of radiofrequency current provided in a central portion of said balloon;
an external radiofrequency generator which supplies radiofrequency energy to said electrode for delivery of radiofrequency current;
a temperature sensor provided inside said balloon;
an external thermometer which detects a temperature of said temperature sensor;
a solution transport path formed between said outer tube and said inner tube, in communication with an inside of said balloon;
an external vibration generator which applies a vibrational wave to an inside of said balloon through said solution transport path; and
a guide wire which guides said balloon to a target site,
wherein said external vibration generator includes an injector comprising an injector outer tube fixed inside a cylinder and an injector inner tube inserted into said injector outer tube, and a piston which is slidably provided inside said cylinder and is driven by a crank coupled to a rotating disk to reciprocate.

2. The radiofrequency thermal balloon catheter system according to claim 1, wherein said injector inner tube is fixed to said piston, and a forward motion of said piston allows the injector inner tube to be pushed to thereby drive out a filling solution in said injector into said solution transport path, causing said balloon to dilate, while a backward motion of said piston allows said injector inner tube to be pulled to thereby draw a filling solution in said solution transport path into said injector, causing said balloon to contract, and besides the amplitude and cycle of said vibrational wave are arbitrarily adjustable depending on a bore diameter of said injector and a rotating speed of said rotating disk.

3. The radiofrequency thermal balloon catheter system according to claim 1, wherein said injector inner tube is not fixed to said piston, with a distance between said injector outer tube and said piston being adjustable, and
wherein a forward motion of said piston allows said injector inner tube to be pushed to thereby drive out a filling solution in said injector into said solution transport path, causing the balloon to dilate, while said balloon contracts in conjunction with a backward motion of said piston, causing a filling solution in said solution transport path to flow into said injector, and besides the amplitude and cycle of said vibrational wave are arbitrarily adjustable depending on a bore diameter of said injector and a rotating speed of said rotating disk, and further driving duration of said filling solution is also arbitrarily adjustable depending on said distance between said injector outer tube and said piston.

4. A radiofrequency thermal balloon catheter system comprising:
a catheter shaft comprising an outer tube and an inner tube which are slidable to each other;
a balloon provided between a distal end of said outer tube and a vicinity of a distal end of said inner tube;
an electrode for delivery of radiofrequency current provided in a central portion of said balloon;
an external radiofrequency generator which supplies radiofrequency energy to said electrode for delivery of radiofrequency current;
a temperature sensor provided inside said balloon,
an external thermometer which detects a temperature of said temperature sensor;
a solution transport path which is formed between said outer tube and said inner tube, in communication with an inside of said balloon;
an external vibration generator which applies a vibrational wave to the inside of said balloon through said solution transport path; and
a guide wire which guides said balloon to a target site,
wherein said external vibration generator includes an injector comprising an injector outer tube fixed to an inside of a cylinder and an injector inner tube inserted into the injector outer tube, and a diaphragm which is fixed inside said cylinder and is driven by a crank coupled to a rotating disk to reciprocate.

5. The radiofrequency thermal balloon catheter system according to claim 4, wherein said injector inner tube is fixed to said diaphragm, and a forward motion of said diaphragm allows said injector inner tube to be pushed to thereby drive out a filling solution in said injector into said solution transport path, causing the balloon to dilate, while a backward motion of said diaphragm allows said injector inner tube to be pulled to thereby draw a filling solution in said solution transport path into said injector, causing said balloon to contract and besides the amplitude and cycle of said vibrational wave are arbitrarily adjustable depending on a bore diameter of said injector and a rotating speed of said rotating disk.

6. The radiofrequency thermal balloon catheter system according to claim 4, wherein said injector inner tube is not fixed to said diaphragm, with a distance between said injector outer tube and said diaphragm being adjustable, and
wherein a forward motion of said diaphragm allows said injector inner tube to be pushed to thereby drive out a filling solution in said injector into said solution transport path, causing the balloon to dilate, while said balloon contracts in conjunction with a backward motion of said diaphragm, causing a filling solution in said solution transport path to flow into said injector, and besides the amplitude and cycle of said vibrational wave are arbitrarily adjustable depending on a bore diameter of said injector and a rotating speed of said rotating disk and further driving duration of said filling solution in said injector is also arbitrarily adjustable depending on said distance between said injector outer tube and said diaphragm.

7. A radiofrequency thermal balloon catheter system comprising:
a catheter shaft comprising an outer tube and an inner tube which are slidable to each other;
a balloon provided between a distal end of said outer tube and a vicinity of a distal end of said inner tube;
an electrode for delivery of radiofrequency current provided in a central portion of said balloon;
an external radiofrequency generator which supplies radiofrequency energy to said electrode for delivery of radiofrequency current;
a temperature sensor provided inside said balloon;
an external thermometer which detects a temperature of said temperature sensor;
a solution transport path which is formed between said outer tube and said inner tube, in communication with an inside of said balloon;
an external vibration generator which applies a vibrational wave to the inside of said balloon through said solution transport path; and
a guide wire which guides said balloon to a target site,
wherein said external vibration generator includes an injector comprising an injector outer tube fixed inside a cylinder and an injector inner tube inserted into said injector outer tube, and a piston-shaped permanent magnet which is slidably provided inside said cylinder and is driven by a magnetic force periodically generated by an electromagnet to reciprocate.

8. The radiofrequency thermal balloon catheter system according to claim 7, wherein said injector inner tube is fixed to said piston-shaped permanent magnet, and the amplitude and cycle of said vibrational wave and a driving duration of a filling solution in said injector are arbitrarily adjustable depending on a magnitude, cycle and duration of a current applied to said electromagnet.

9. The radiofrequency thermal balloon catheter system according to claim 7, wherein a distance between the injector outer tube and said piston-shaped magnet is adjustable, and the amplitude and cycle of said vibrational wave and a driving duration of the filling solution in said injector are arbitrarily adjustable depending on a magnitude, cycle and duration of a current applied to said electromagnet.
